(19) United States
(12) Patent Application Publication
(10) Pub. No.: US 2017/0055870 A1
(43) Pub. Date: Mar. 2, 2017

(54) Simplified ECG Acquisition, Transmission And Sharing System

(71) Applicant: Spaulding Medical, LLC, West Bend, WI (US)

(72) Inventors: Randol R. Spaulding, West Bend, WI (US); Joseph Angelo Erato, West Bend, WI (US); Shaun Szot, Milwaukee, WI (US); Nick Fetcko, West Bend, WI (US); Amanda Baltz, Shorewood, WI (US)

(21) Appl. No.: 15/246,171

(22) Filed: Aug. 24, 2016

Related U.S. Application Data
(60) Provisional application No. 62/209,464, filed on Aug. 25, 2015.

(51) Int. Cl. A61B 5/0428; A61B 5/0404; A61B 5/0452; A61B 5/04; A61B 5/0408; A61B 5/00; A61B 5/044

(52) U.S. Cl. CPC A61B 5/04288; A61B 5/0006; A61B 5/0404; A61B 5/044; A61B 5/749; A61B 5/04017; A61B 5/0408; A61B 5/0452; A61B 2562/227

(57) ABSTRACT

An electrocardiogram (ECG) acquisition and viewing system receiving extended ECG recordings and automatically extracting segments with heart arrhythmias allowing overreading cardiologists to quickly analyze the extract segments from the larger data set. The present invention provides an easy to use, lightweight ECG module providing acquisition of 12 lead ECG data and communication with local and remote network devices receiving the ECG data The present invention provides wireless communication (e.g., Bluetooth, near field communication) with local devices so that ECG waveforms may be viewed in real time, allowing lead connections to be corrected and integrity restored before and during data acquisition.

### CROSS-REFERENCE TO RELATED APPLICATION

This application claims the benefit of U.S. provisional application Ser. No. 62/209,464 filed Aug. 25, 2015 and hereby incorporated by reference.

### BACKGROUND OF THE INVENTION

1. Field of the Invention

The present invention relates to electrocardiogram (ECG) acquisition and viewing systems and, more specifically, to an ECG module receiving 12 lead ECG data from a patient and communicating wirelessly with a computer device for displaying the ECG data and uploading ECG data to a network patient database.

2. Discussion of the Related Art

ECG acquisition and viewing systems allow medical professionals to monitor electrical activity of the heart in real time and for extended periods of time Normally, cardiac rhythms are obtained from surface ECG electrodes placed on a patient's chest and the ECG signals are then viewed by the medical professionals on bedside monitors. Once the ECG signal is viewed on the monitors, adjustments may be made to the placement or attachment of the surface ECG electrodes to achieve a better signal. Monitors may also include built-in signal filters to help create clean waveforms eliminating unwanted noise and artifact. The recordings are typically recorded for short intervals for later interpretation by the overreacting cardiologist. The typical 12 lead ECG is a ten□ second recording, most often displayed in a three□ by□ four format, which provides 2.5 seconds of waveform data for each lead. Given the considerable biological variability of the ECG, which occurs on a beat□ to□ beat basis and over longer cycle times, and the often more striking variability associated with acquisition and processing of the body□ surface signal, traditional ECG does not provide the most comprehensive “electrocardiographic state” of the patient, often missing vital arrhythmias,

While many 12-lead ECGs are capable of making longer recordings, requiring adequate electronic storage and on/off functionality, these systems are typically unable to handle the extended data in a way which assists the physicians in making improved 12-lead ECG diagnostic statement capabilities.

### SUMMARY AND OBJECTS OF THE INVENTION

The present invention provides an electrocardiogram (ECG) acquisition and viewing system receiving extended, e.g. five□ minute, ECG recordings and automatically extracting segments with heart arrhythmias allowing overreading cardiologists to quickly analyze the extracted segments from the larger data set. The extended recordings are able to detect sporadic arrhythmias and arrhythmias (i.e., heart rate variability, QTc variability, and T-wave morphological variability) which are risk indicators for cardiovascular and sudden death that are often not captured on traditional ten-second ECGs. Extended ECGs eliminate the need for triplicate recordings, a commonplace method used to reduce the influence of biological and methodological variability of the ECG but now unnecessary with extended recordings.

The present invention provides an easy to use lightweight ECG module providing acquisition of 12 lead ECG data and communication with network devices receiving the ECG data. By storing the extending recording, the ECG may be acquired anywhere, even where network communication is unavailable, and uploaded to a medical database server at a later time when network communication is restored.

The present invention provides wireless communication (e.g., Bluetooth, near field communication) with local devices so that ECG waveforms may be viewed in real time, allowing lead connections to be corrected and integrity restored before and during data acquisition.

One embodiment of the present invention provides an electrocardiograph (ECG) module for receiving ECG data and delivering the ECG data to an electronic computer, the ECG module having a portable housing; a port located within the housing and adapted to receive a connector for receiving the ECG data from a plurality of ECG electrical leads; a single button accessible to the user on the housing for operating a module control program to activate receipt of the ECG data and a communication device for communicating the ECG data to the electronic computer.

The ECG module may include a display screen displaying a graphical representation of lead integrity.

The ECG module may include a display screen displaying a graphical representation of battery life. The ECG module may include a microphone adapted to record sound waves.

The ECG module may include an audio processor adapted to receive the sound waves and convert the sound waves to digital audio signals of a user's voice.

The ECG module may include a USB port for wired connection to the electronic computer.

The ECG module may include a Bluetooth radio adapted to communicate with a near field communication device of the electronic computer. The electronic computer may be a portable computing device. The portable computing device may deliver the ECG data to a server and a second electronic computer may communicate with the server to receive the ECG data over the Internet.

Another embodiment of the present invention provides a method of acquiring electrocardiograph (ECG) data, the method including the steps of: connecting ECG electrodes to a patient; receiving extended duration ECG data from the ECG electrodes at a module; transmitting the ECG data to a network server of a network; acquiring the ECG data at a device communicating with the network server; reviewing the ECG data at the device; and producing a report representative of the ECG data.

The method may further include generating filtered ECG data from the raw ECG data at the module network server.

The method may further include transmitting the ECG data from the module to an electronic computer via a communication protocol and viewing the ECG data at the electronic computer in real time. The communication protocol may be near field communication. The electronic computer may be a portable computing device.

The method may further include notifying a medical professional that ECG data has been transmitted to the network server.

The method may further include receiving the ECG data from the network server and automatically identifying segments of the ECG data identifying heart arrhythmia.

The method may further include modifying the report for the ECG data.

The report may include a medical professional's interpretation of the ECG data.

The network server may store previous ECG data of the patient. The report may include comparison of previous and current ECG data

These and other aspects and objects of the present invention will be better appreciated and understood when considered in conjunction with the following description and the accompanying drawings. It should be understood, however, that the following description, while indicating representative embodiments of the present invention, is given by way of illustration and not of limitation. Many changes and modifications may be made within the scope of the present invention without departing from the spirit thereof, and the invention includes all such modifications.

### BRIEF DESCRIPTION OF THE DRAWINGS

A clear conception of the advantages and features constituting the present invention, and of the construction and operation of typical mechanisms in accordance with the present invention, will become more readily apparent by referring to the exemplary, and therefore non-limiting, embodiments illustrated in the drawings accompanying and forming a part of this specification, wherein like reference numerals designate the same elements in the several views.

In the drawings:

FIG. 1 is a block diagram of a ECG system per the present invention providing communication between an ECG module, computer devices, and a medical database server showing various functional elements of the module including an electronic controller executing a control program;

FIG. 2 is a top plan view of the ECG module including a display and single push button;

FIG. 3 is a side elevation view of the ECG module showing connection ports of the ECG module;

FIG. 4 is a perspective view from above of the ECG module connected to a 12 lead ECG wire;

FIG. 5 is a depiction of a tablet computer displaying real time ECG waveform data and including an electronic controller executing a control program;

FIG. 6 is a depiction of a desktop computer displaying a diagnostic report and including an electronic controller executing a control program:

FIG. 7 is a flowchart showing the steps implemented with respect to FIGS. 1 and 2 implemented by an executed program on the ECG module; and

FIG. 8 is a flowchart showing the steps implemented with acquiring ECG data from a patient.

In describing the embodiments of the invention which are illustrated in the drawings, specific terminology will be resorted to for the sake of clarity. However, it is not intended that the invention be limited to the specific terms so selected and it is to be understood that each specific term includes all technical equivalents which operate in a similar manner to accomplish a similar purpose. For example, the word connected, attached, or terms similar thereto are often used. They are not limited to direct connection but include connection through other elements where such connection is recognized as being equivalent by those skilled in the art.

### DETAILED DESCRIPTION OF THE INVENTION

The various features and advantageous details of the subject matter disclosed herein are explained more fully with reference to the non-limiting embodiments described in detail in the following description.

### The ECG Sharing System

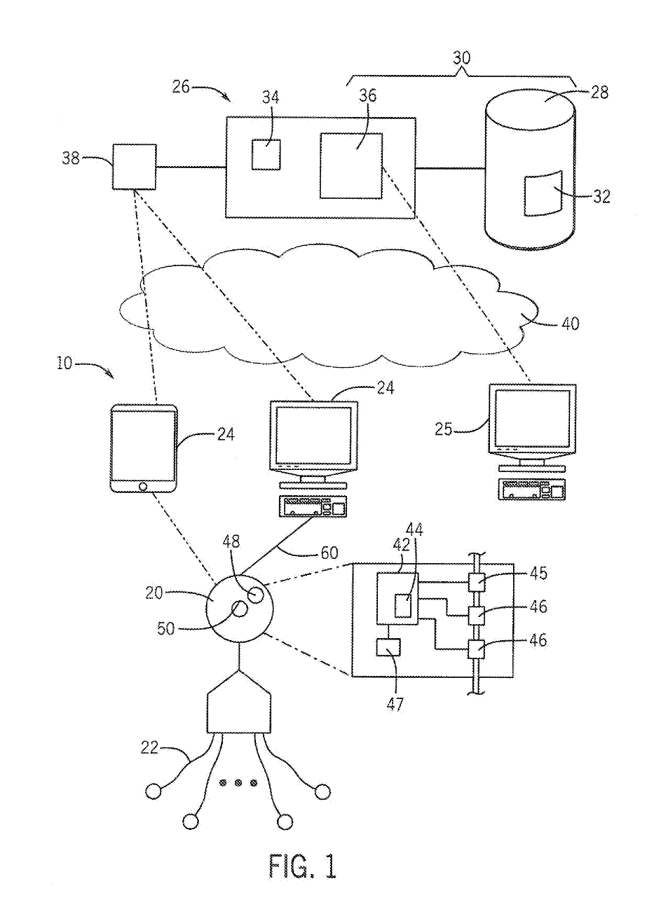

Referring to FIG. 1, an electrocardiogram (ECG) system 10 using an ECG module 20 is provided which interfaces with typical ECG lead wires 22 for real-time acquisition and display of ECG heart rhythm data on a device, which may be in the form of an electronic computer 24 such as a desktop computer, cell phone, PDA, tablet or the like, and synchronization to a network 40 for further review, analysis and/or storage. The ECG system 10 may be implemented in a hospital, clinic, ambulance, or home environment or other remote location, providing improved mobility and flexibility to the user, such as a clinician or trained medical professional, acquiring the ECG data from a patient.

The ECG system 10 includes an ECG server system 26 that may be part of a standard hospital electronic medical record system and may include a memory system 28, for example, providing a disk array or the like. The memory system 28 may provide part of an electronic medical database 30 holding medical information and patient records and may include a database table 32 providing ECG information linked to particular patient identification, such as a patient's identification number or name, date of birth, and voice print. Alternatively, the ECG server system 26 may be separate from the standard hospital electronic medical record system and the information stored in the database table 32 may be uploaded to the hospital electronic medical record system's separate memory.

It will be understood that the database 30 provides both file structures on physical non-transient medium and also a program or database engine for accessing that data according to query instructions. In this regard, the database 30 may provide for a standard database interface, for example, using standard query language or a standardized API, and may further provide an interface accessible over a network. In one embodiment, the network interface may allow communication with the standard database interface using standard network interface conventions, for example, as may be implemented under HTML, XML or other well-known standards. The database engine and portions of the database 30 may be implemented by an electronic computer 34 being part of the ECG server system 26 executing a stored program 36 contained therein.

The ECG server system 26 may communicate with a wireless network circuit 38 or the like that may implement a portion of the network 40, for example, providing standard wireless communication protocols such as IEEE 802.11 (a)/(b)/(g)/(n). The wireless network card may in turn communicate with corresponding wireless circuitry in each of the electronic computers 24 as will be discussed below. The network 40 may also include physical media such as optical or electrical conductors.

The ECG server system 26 may connect via the network 40 with other computer devices 25 for use in a hospital or other health care setting. The ECG server system 26 may also connect with computer devices 25 that are remotely located, such as outside of the network 40. The computer devices 25 such as a desktop computer, cell phone, PDA, tablet or the like may access the database 30 through a mobile app, web app or a standard browser program to retrieve particular information from the database 30, such as ECG data for a particular patient from a physician's ECG order.

The computer device 25 may access the database 30 from outside the network 40 or from a remote network by pulling the requested information of the electronic medical database 30 from the network 40 once access has been granted. Alternatively, access may be granted for the requested data from the database 30 to be copied to the remote network storage (not shown) and retrieving the requested data from the remote network database.

The ECG module 20 may connect to an internal controller 42 and execute a stored program 44 fixed on physical non-transient medium to provide control 45 of the ECG module 20 according to the program 44 and according to the readings of the sensors 46, which may sense the connection of the ECG lead wires 22, the connection of a charging cord, and/or the quality of the transferred data. The controller 42 may also communicate with user interface elements of the module 20 including a display screen 48 and an actuator, such as a button 50 or the like.

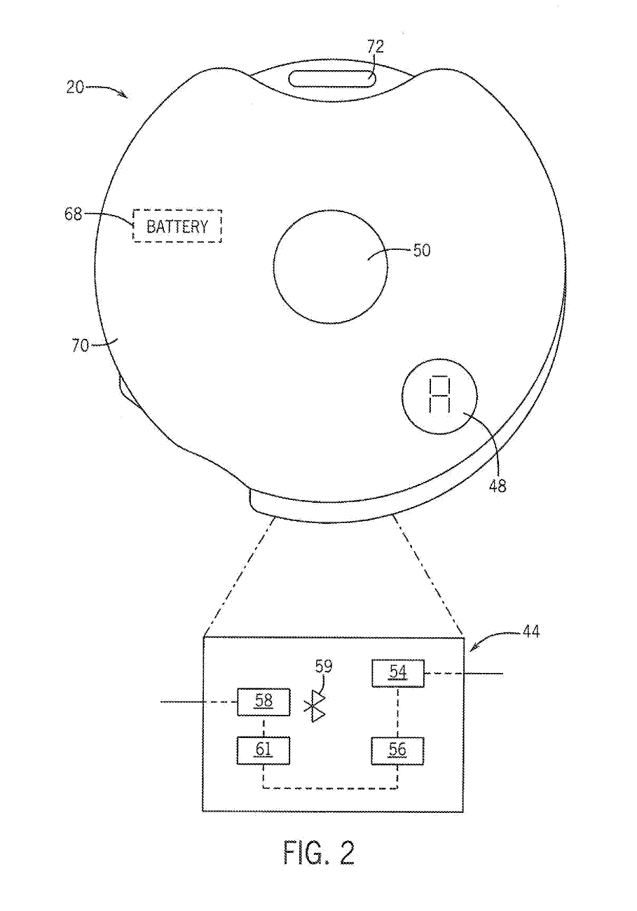

Referring also to FIG. 2, the ECG module 20 may include a program 44 to provide a number of program elements including a module control program 54, voice processing 61, and communication protocol stack 58, to be further described below.

The module control program 54 receives a control signal from the push button 50 to activate and stop receipt of ECG heart rhythm data from the ECG lead wires 22. The module control program 54 also communicates with user interface routines 56 allowing receipt of data from and transmission of data to the user interface formed by the display screen 48. The data may include indications that the module 20 is ready to record a new ECG, ready to capture voice print, and ready to upload ECG data to network. The data may also include battery life data, Bluetooth pairing data, and charging data also provided on the display screen 48.

The program 44 may further include voice processing 61 for receiving a patient's voice sound waves via a microphone 65 and converting the sound waves to digital audio signals indicating the voice frequency, duration and amplitude of the patient's voice, which is then uploaded to the electronic medical database 30 and included in the patient's medical record. The audio signals may be viewed by a computer 24, 25 as a spectrogram or a voice print of the patient's voice. Thus, audio signals or voice prints from a subsequent voice recording may be compared to pre-saved audio signals or voice prints to provide biometric matching and verification of a patient's identity.

The program 44 may further include a communication protocol stack 58, e.g., Bluetooth, near field communication, infrared channel communication, or the like, for communication with the paired electronic computer 24 for receipt of data therefrom and transmission of data thereto. The wireless communication between the ECG module 20 and the paired electronic computer 24 may be Bluetooth communication, which is compatible over a number of platforms including Apple, Android and Microsoft, or may be any other satisfactory communication protocol for wireless transmission of data. The Bluetooth communication may be established via Bluetooth radios 59 in the respective devices. In order to enable Bluetooth communication, the ECG module 20 may need to be paired with the receiving paired electronic computer 24 to be further described below. If no Bluetooth communication can be established, the ECG data will be saved on a memory device 47 of the ECG module 20 having the capability to store an extended duration, e.g., at least five minutes, of ECG data, until a Bluetooth connection can be established or until a wired connection 60 can be established between the ECG module 20 and the electronic computer 24.

Still referring to FIG. 2, the ECG module 20 may include a round, puck-shaped housing 70 that may be made of a lightweight durable material, such as, for example, a plastic, polycarbonate, or acrylnitrile-butadiene-styrene. The housing 70 may also be made of a durable, non-plastic material. The housing 70 may be easily cleaned with a dry cloth. The housing 70 is portable and sized to fit within a standard pocket so that the module 20 may be conveniently stored within the user's pocket before, during, and after use of the ECG module 20. The housing 70 may be, e.g., approximately 2-5 inches in diameter by 0.5-2 inches wide. The housing 70 may be lightweight, e.g., approximately 2-5 ounces. The housing 70 may include a slot 72 to attach the housing 70 to a lanyard or clip. The ECG module 20 may contain a rechargeable battery 68 stored within the housing 70. The housing 70 may include a single switch such as the push button 50 and the display screen 48 such as a LED screen to be further described below

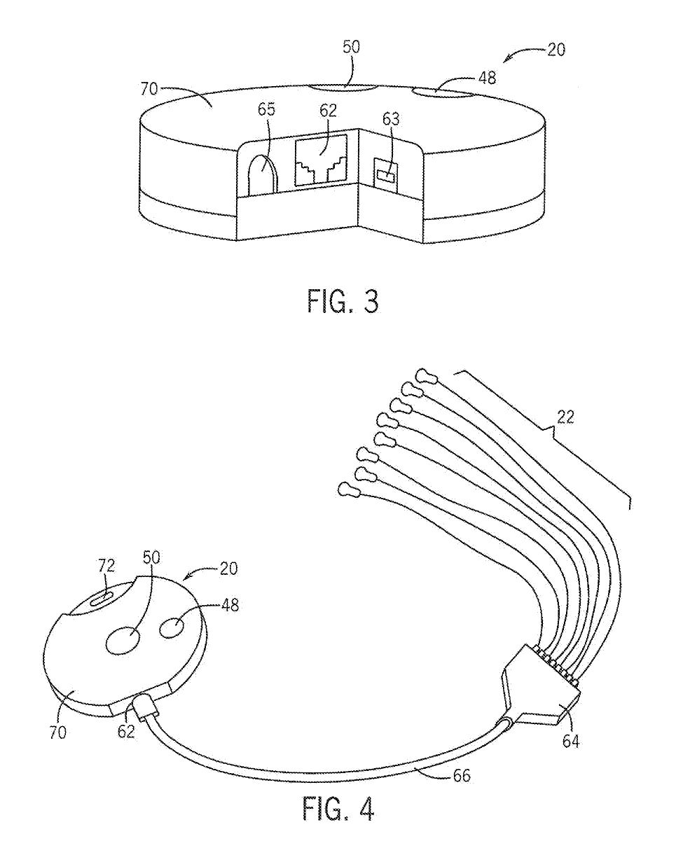

Referring to FIGS. 3-4, the ECG module 20 may include a number of ports for receiving electrical connectors. An ECG cable port 62 may connect the plurality of ECG lead wires 22 via a common yoke connector 64 combining the lead wires 22 into a single trunk cable 66. The single trunk cable 66 may then be connected to the ECG cable port 62 for receipt of ECG data. It is contemplated that the ECG module 20 may accept conventional 12-lead ECG data, however, it may also accept 1-lead, 3-lead or 5-lead ECG data. A port 63, such as a USB or mini-USB port, may connect to a connector of an external power source (not shown), such as utility power or external battery supply, for charging and recharging the ECG module's internal battery 68. The port 63 may also be used to provide a wired connection 60 between the ECG module 20 and the electronic computer 24 for upload of ECG data to the electronic computer 24. The housing 70 may also include a microphone 65 for receiving a voice sample from the patient. The ECG module 20 may utilize the single push button 50 toward its center for easy operation of the device. It is contemplated that the button 50 may also take other forms of switches, such as a toggle switch or slide. The push button 50 may be used to send a control signal to switch the ECG module 20 ON and OFF. For example, the ON/OFF operation may require a long press of the push button 50 for turning the module 20 ON and OFF.

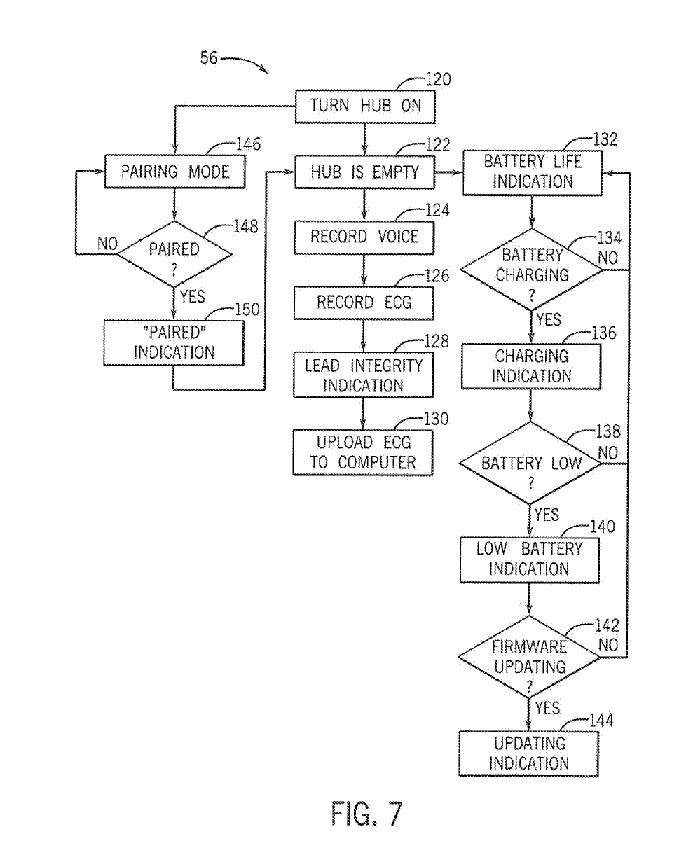

Referring also to FIG. 7, the display screen 48 of the ECG module 20 may be an LED screen using intuitive display characters to allow for ease of operation of the device while going through a number of user interface routines 56. For example, once the ECG module 20 is switched ON, as indicated by process block 120, the display screen 48 may display an “E” indicating that the device is empty and ready to acquire a new ECG, as indicated by process block 122. Once the button is pressed again, the display screen 48 may display an “R” indicating that the device is ready to record a voice sample of the patient via the microphone 65 of the ECG module 20, as indicated by process block 124. The patient may then recite a common phrase, such as the patient's own name, into the microphone 65. Once the patient's voice is properly recorded, the display screen 48 may display the letter “A” indicating that the device is ready to record an ECG, as indicated by process block 126.

Once the ECG lead wires 22 are attached to the ECG module 20 as described above, the display screen 48 may display a number of bars indicating the lead integrity (e.g., indicating lead fractures) or connection quality of the device so that the user may correct any poor connections of the lead wires 22 or single trunk cable 66 ore replace any broken wires, as indicated by process block 128. When the ECG is finished recording or the recording is stopped by a button 50 press, the display screen 48 may display the letter “F” indicating that the ECG is ready to be uploaded to the electronic computer 24, as indicated by process block 130.

The push button 50 may also be used to activate other user interface routines 56. For example, a single short press of the ECG module 20 may activate a battery life indicator on the display screen 48 for displaying a digital or graphical representation of the remaining battery life of the device, as indicated by process block 132. For example, the display screen 48 may present a number between 0-9 indicating a battery level. The display screen 48 may also show other visual indicators to assist the user during battery charging, such as displaying a “C” indicating that the device is charging whereas the “C” would disappear when the device is fully charged, as indicated by process block 134 and 136. Also, a “L” may show on the display screen 48 when the ECG module's 20 battery 68 is too low to receive and transmit ECG data, as indicated by process block 138 and 140. In addition, a “U” may indicate that the firmware of the ECG module 20 is being updated so that the user does not remove the USB cable during updating, as indicated by process block 142 and 144.

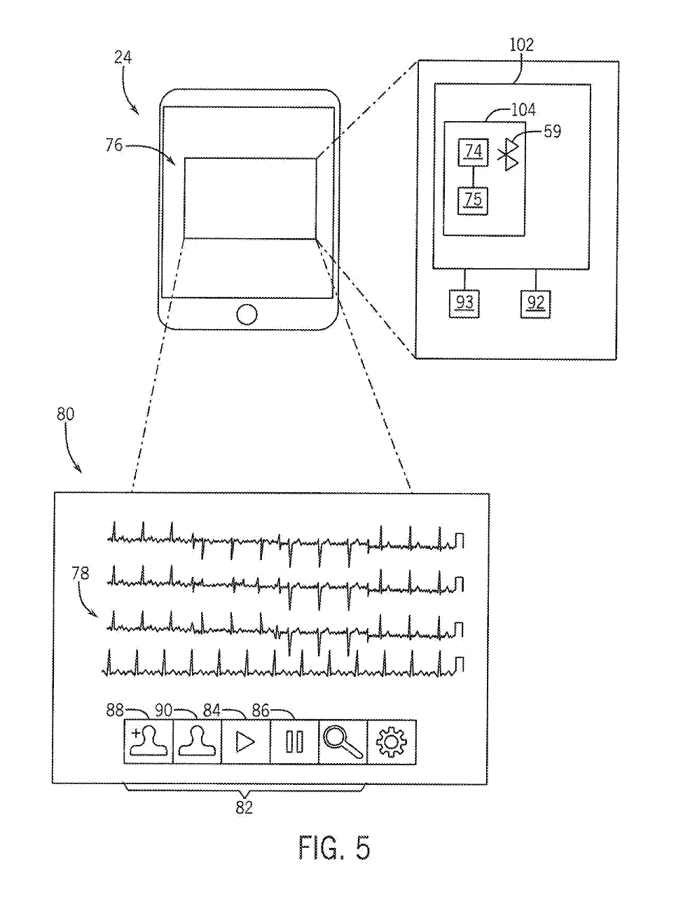

Referring also to FIG. 5, the push button 50 may also be used for initial pairing of the ECG module 20 to an electronic computer 24, e.g., via Bluetooth. The button 50 may be pressed and held until a “P” is shown on the display screen 48 indicating that the module 20 is in pairing mode, as indicated by process block 146. A “P.” (with a period) shown on the display screen 48 would then indicate that the ECG module 20 has been successfully paired, as indicated by process blocks 148 and 150.

The Bluetooth paired electronic computer 24 may include an internal controller 102 and execute a stored program 104 fixed on physical non-transient medium to provide control of the paired electronic computer 24 according to the program 104. The program 104 may include a communication protocol stack 74 similar to the Bluetooth protocol stack 58 of the ECG module 20 described above for receiving real time ECG data from the ECG module 20. The Bluetooth communication may be established via a Bluetooth radio 59 of the electronic computer 24. The paired electronic computer 24 may include a mobile app 76 or other computer or browser program for filtering the raw ECG data and displaying the filtered ECG data waveforms 78. For example, the mobile app 76 may provide a user interface 80 including a graphical representation of the filtered ECG data waveforms 78.

The user interface 80 may also include user functions 82 for operating ECU data retrieval and patient information. For example, the user interface 80 may include a START icon 84 to initiate saving ECG data to the paired electronic computer 24. The interface 80 may also include a PAUSE icon 86 to stop saving ECG data to the paired electronic computer 24. The interface 80 may include the ability to add a new patient 88 or enter patient demographics 90 for the patient whose ECG data is being received. The ECG data and patient data may be saved to a memory device 93.

The program may further include a network stack 75 for providing wireless communication with the network via a wireless network circuit 92 similar to that described above for communication over the network 40. The mobile app 76 may display a message to the user of whether to upload the acquired ECG data to the ECG server system 26 once the data capture is complete, for example once the PAUSE icon 86 is pressed or once the ECG data is uploaded from the ECG module 20 through a wired connection 60. e.g., USB cable. The user may confirm patient demographics, or add new patient demographics, allowing the ECG data to be linked to particular patient information, such as a patient identity, and the ECG data will be transferred automatically to the ECG server system 26 via the wireless network circuit 38 when a proper network connection can be established. If no network connection can be established, the ECG data will be saved on the paired electronic computer 24 until a connection is restored. In the meantime, the user may continue to save ECG data to the paired electronic computer 24.

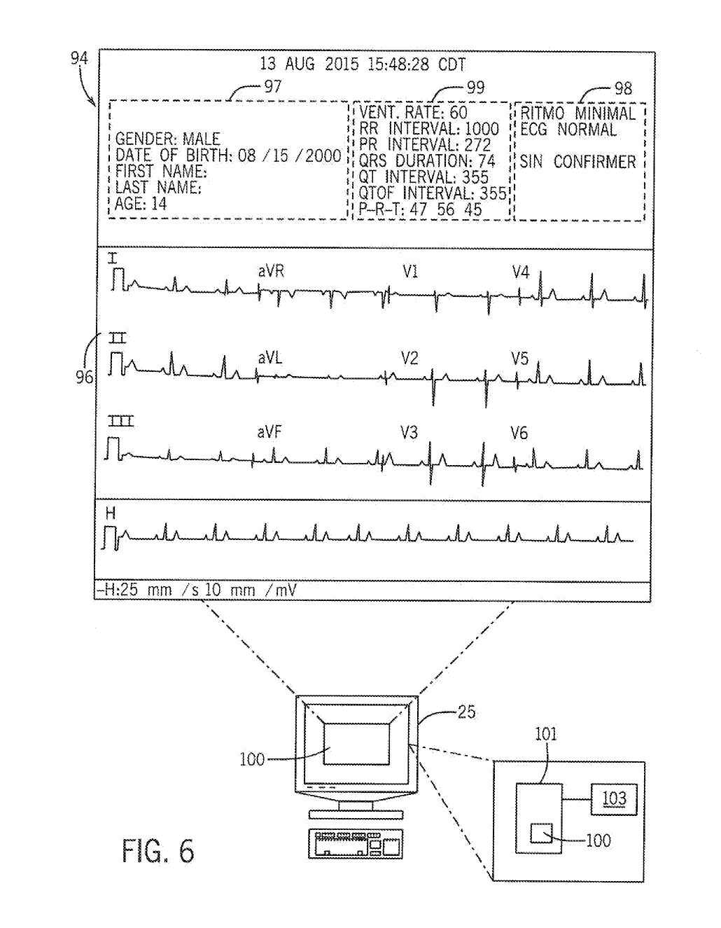

Referring to FIGS. 1 and 6, when a network connection is established by the paired electronic computer 24, the ECG data is communicated to the ECG server system 26, which may be processed by the stored program 36 via algorithms or other third party tools to automatically analyze the acquired data. For example, the program 36 may assess morphology and cardiac changes and extract segments containing cardiac arrhythmias from the ECG recording. The analysis may consider a selected time frame and duration of the ECG data, such as between ten seconds to ten minutes of ECG data recordings. The analysis may then be presented in a diagnostic report 94 of the patient's ECG data, which may provide a snapshot of the ECG waveform 96 along with an initial diagnosis 98 of the ECG, such as normal. sinus tachycardia, sinus bradycardia, sinus arrhythmia, and the like. Other information may be found on the diagnostic report 94 such as patient information 97 and details 99 of the selected ECG waveform 96. The stored program 36 may also perform signal filtering of the ECG data for filtering the ECG heart rhythm data and eliminating unwanted interference found in the raw signals, such as baseline drift.

The ECG system 10 allows a computer device 25, either connected to the local network 40 or connected to a remote network or to the network over the Internet, to retrieve the diagnostic report 94 from the memory system 28 of the ECG server system 26. For example, a physician (i.e., overreading cardiologist) working on a computer device 25 communicating from either inside the local network 40 or outside the local network 40, such as a remote network, may retrieve the diagnostic report 94 from a mobile app, web app or a standard browser program 100 on the computer device 25, such as a desktop computer, cell phone, PDA, tablet or the like. The computer device 25 may include an internal controller 101 and execute the stored program 100 fixed on physical non-transient medium and stored in memory 103 to provide control of the computer 25 according to the program 100. The physician may then log in to the secure system, e.g., using a username and password, and confirm or edit the diagnostic report 94 remotely. The confirmation and/or edits by the physician are stored in the memory system 28 and saved as part of the diagnostic report's 94 history. The physician may also add notes to the diagnostic report 94, which are also saved as part of the history of the diagnostic report 94. Previously acquired ECG data may also be compared for revealing interval and morphology changes between ECGs taken at different times. The diagnostic report 94 may include a variety of information including summary, selected, and comparison ECGs, beat data, interval values, diagnostic statements, heart rate variability, QT variability, T wave morphology, signal average ECG, P-R-T axes and the physician's final interpretation of the ECG.

### Method of Use

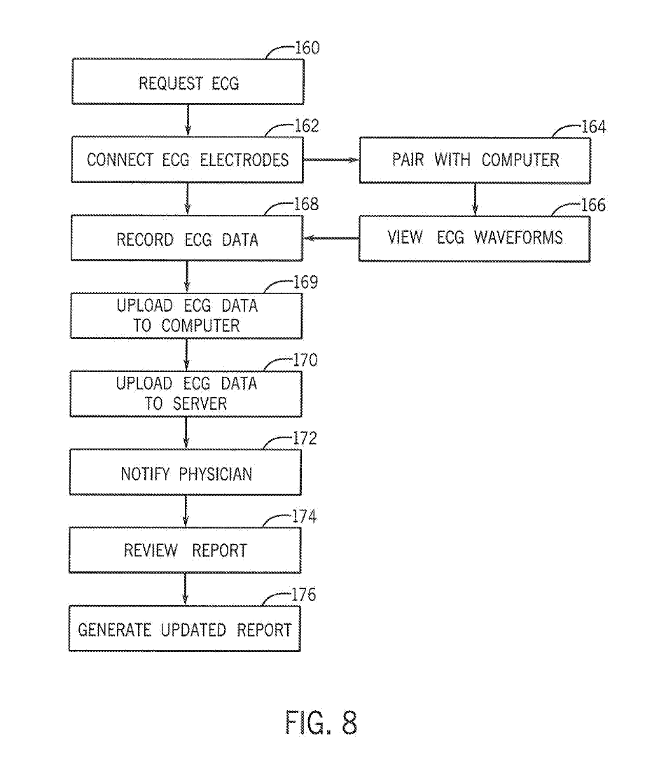

Referring now to FIGS. 1 and 8, the workflow for a diagnostic ECG exam using the ECG system 10 typically starts with a physician's order requesting the ECG exam be done on the patient as indicated by process block 160. Alternatively, the ECG exam may be conducted by the patient himself or herself, or by a technician, typically with prior authorization of a physician. To administer the ECG exam a plurality of ECG electrodes coupled to the ECG lead wires 22 are placed at designated areas of the patient's body, as known in the art. The single trunk cable 66 delivering the data transmitted by the ECG lead wires 22 is inserted into the ECG cable port 62 of the ECG module 20 so as to establish communication between the ECG lead wires 22 and the ECG module 20, as indicated by process block 162.

Optionally, the ECG module 20 is paired to the electronic computer 24 to establish Bluetooth communication between the devices, as indicated by process block 164. The ECG module 20 may be paired by pressing and holding the push button 50 until a “P” is shown on the display screen 48 of the ECG module 20 indicating pairing mode is ON. The electronic computer 24 should also be placed in pairing mode. When “P.” is shown on the display screen 48 of the ECG module 20, it is confirmed that the ECG module 20 and the electronic computer 24 have been successfully paired.

Once the ECG module 20 and paired electronic computer 24 are paired, the ECG module 20 may be turned ON and the mobile app 76 opened. The mobile app 76 can verify the connection of the paired electronic computer 24 with the ECG module by displaying real time streaming EGG data waveforms 78 on the mobile app's user interface 80, as indicated by process block 166.

The user may view the waveforms 78 and identify poor lead quality or connection, such as viewing interference causing noise in the waveforms or square waves indicating a broken or disconnected lead. The user interface 80 may display a message, such as “Leads Off/Not Appearing” if there are leads that are not attached appropriately to the patient. The user may correct for poor lead quality or connection prior to saving the ECG data by adjusting the leads/connectors or replacing broken equipment. The user interface 80 may also display a message indicating that the ECG module 20 is not properly paired with the electronic computer 24, for example, if the electronic computer 24 is paired incorrectly or the ECG module 20 is out of range.

Lead connection indicators may also be shown on the display screen 48 of the ECU module 20 indicating the lead integrity strength, for example, displayed as bars with more bars indicating greater strength, so that the user may correct the lead connections prior to ECG recording.

Once the waveforms 78 and/or lead integrity strength are confirmed by the user, he or she may use the START icon 84 on the electronic computer 24 to begin saving the ECG data to the electronic computer 24, or press the button 50 of the ECU module 20 to begin saving the ECG data to the ECG module 20, as indicated by process block 168.

if the electronic computer 24 is paired, a counter may provide a visual indication of the amount of time recorded or the amount of data recorded. Once the user presses the PAUSE icon 86, the saving is stopped. The user interface 80 may then display a message asking the user whether he or she would like to transfer the ECG data to the ECG server system 26. Once the user chooses to upload the ECG data to the ECG server system 26, the user may be asked to select the ECG physician's order associated with the capture and/or the patient demographics for the ECG data. If the patient demographics are not already saved in the system, the user may be asked to enter the patient as a new patient. Once an Internet connection is established, the ECG data is automatically uploaded to the ECG server system 26, as indicated by process block 170, and the data may be deleted from the memory of the electronic computer 24.

If the electronic computer 24 is not paired, the display screen 48 of the ECG module 20 will indicate that ECG data acquisition is complete when a certain amount of time has been recorded, e.g. five minutes, a certain amount of data has been recorded, e.g., when the memory is near full, or when the button 50 has been pressed to stop recording. The display screen 48 will indicate an “F” indicating that it is ready to upload to the ECG data to the electronic computer 24. The ECG data may also be uploaded to the electronic computer 24 via a wireless or wired connection. Once the ECG data is uploaded to the electronic computer 24, as indicated by process block 169, the data may be deleted from the memory of the ECG module 20. Then, the ECG data may be uploaded from the electronic computer 24 to the ECG server system 26, as indicated by process block 170, and the data may be deleted from the memory of the electronic computer 24.

The stored program 36 of the ECG server system 26 may perform some initial processing of the ECG data to automatically analyze the acquired data such as assess morphology and cardiac changes and extract segments containing cardiac arrhythmias from the ECG recording, as indicated by process block 172. Thus, shorter segments of the extended ECG data may be highlighted so that the physician may view areas of concern without viewing the entire data set. The identified segments may be provided to the reviewing physician in the generated ECG diagnostic report 94. The stored program 36 may also provide a computer-derived diagnosis to be reviewed by the physician in the ECG diagnostic report 94. The stored program 36 may also perform filtering of the ECG data for filtering the ECG heart rhythm data and eliminating unwanted interference found in the raw signals, such as baseline drift.

Automated notifications may be provided to the patient's healthcare team whenever ECG data is delivered to the ECG server system 26 so that the patient's physician may review the generated ECG diagnostic report 94 without delay, as indicated by process block 172. For example, the notifications may appear on the physician's mobile phone having the mobile app or program 100 downloaded thereon. Alternatively, an email or text notification may be sent to the physician's mobile phone indicating a new uploaded ECG. The notifications may also be sent to other actors within the healthcare system, such as electronic medical records and medical billings departments.

Once the ordering physician is notified that ECG data has been uploaded to the ECG server system 26, he or she may view the diagnostic report 94 produced by the ECG server system 26, as indicated by process block 174. The physician may log in to their account on the mobile app, web app or a standard browser program 100 on their computer device 25 using login credentials, such as a username and password. The program 100 may provide a queue of unconfirmed or unviewed ECG diagnostic reports 94 for the physician to review. The physician may select the diagnostic report 94 to view the report. Zoom and scroll abilities may assist the physician in viewing the report. The physician may view the diagnostic report 94 and confirm the diagnosis 98 or edit the diagnosis. The physician may make additional notes, which are added to the record. All changes or edits are saved into the record as part of the transaction history and creating an updated report. The physician may use the program 100 to view diagnostic reports 94 by searching by patient identification or by physician order identification. The physician may also send or email the report 94 in various formats, such as .pdf or .png files, or send the report to a printer for printing, as indicated by process block 176.

While the invention contemplates the capture of ECG data, it is possible that a similar system may be used for the capture of other human vital data, such as but not limited to blood pressure, body temperature, pulse rate, respiratory rate, peripheral capillary oxygen saturation and the like to the hub 20. In a similar manner, the human vital data is captured by a medical device and transmitted to the hub 20 for communication with the electronic computer 24. The human vital data can then be uploaded to a server system 26 for retrieval by remote computers, such as computer device 25. The human vital data may be obtained by leads that are connected to hub 20 separately from or along with the ECG lead wires 22. Human vital data may also be captured along with the ECG data by transmitting the data to the electronic computer 24, which can then be uploaded to the server system 26. The human vital data can also be viewed in the diagnostic report 94.

The ECG module 20 improves the availability for clinicians or trained health care personnel to acquire, process, display, store, and/or print diagnostic ECG reports for a licensed physician to view, analyze, interpret and/or verify. In this respect, the ECG module's small and compact design allows the ECG module to be easily shipped and transported. During use, the module may be discreetly transported such as within the user's pocket.

The single button 50 design of the ECG module 20 also simplifies operation and the acquisition process. The user simply needs to turn on the ECG module 20 and the mobile app 76 or computer program to initiate acquisition of ECG data. The easy operation of the module simplifies the training required for operation of the module and reduces the chances of human error.

ECG data may be uploaded to the network 40 via a wireless connection. Thus, the ECG data may be uploaded at remote sites, such as clinics, ambulances, and homes, where the physician is not located. Then, when ECG data is uploaded to the network 40, it is accessible to the physician anywhere, including remote sites. By storing the ECG data at the network 40, the efficiency at which ECG diagnostic reports 94 may be retrieved and confirmed by the physician is streamlined.

It should be understood that the invention is not limited in its application to the details of construction and arrangements of the components set forth herein. The invention is capable of other embodiments and of being practiced or carried out in various ways. Variations and modifications of the foregoing are within the scope of the present invention. It also being understood that the invention disclosed and defined herein extends to all alternative combinations of two or more of the individual features mentioned or evident from the text and/or drawings. All of these different combinations constitute various alternative aspects of the present invention. The embodiments described herein explain the best modes known for practicing the invention and will enable others skilled in the art to utilize the invention.

Certain terminology is used herein for purposes of reference only, and thus is not intended to be limiting. For example, terms such as “upper”, “lower”, “above”, and “below” refer to directions in the drawings to which reference is made. Terms such as “front”, “back”, “rear”, “bottom” and “side”, describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first”, “second” and other such numerical terms referring to structures do not imply a sequence or order unless clearly indicated by the context.

When introducing elements or features of the present disclosure and the exemplary embodiments, the articles “a”, “an”, “the” and “said” are intended to mean that there are one or more of such elements or features. The terms “comprising”, “including” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

References to “a microprocessor” and “a processor” or “the microprocessor” and “the processor,” can be understood to include one or more microprocessors that can communicate in a stand-alone and/or a distributed environment(s), and can thus be configured to communicate via wired or wireless communications with other processors, where such one or more processor can be configured to operate on one or more processor-controlled devices that can be similar or different devices. Furthermore, references to memory, unless otherwise specified, can include one or more processor-readable and accessible memory elements and/or components that can he internal to the processor-controlled device, external to the processor-controlled device, and can be accessed via a wired or wireless network.

It is specifically intended that the present invention not be limited to the embodiments and illustrations contained herein and the claims should be understood to include modified forms of those embodiments including portions of the embodiments and combinations of elements of different embodiments as come within the scope of the following claims. All of the publications described herein, including patents and non-patent publications are hereby incorporated herein by reference in their entireties.

We claim:

1. An electrocardiograph (ECG) module for receiving ECG data and delivering the ECG data to an electronic computer, the ECG module comprising:
a portable housing;
a port located within the housing and adapted to receive a connector for receiving the ECG data from a plurality of ECG electrical leads;
a single button accessible to the user on the housing for operating a module control program to activate receipt of the ECG data: and
a communication device for communicating the ECG data to the electronic computer.

2. The ECG module of claim 1 further comprising a display screen displaying a graphical representation of lead integrity.

3. The ECG module of claim 2 further comprising a display screen displaying a graphical representation of battery life.

4. The ECG module of claim 1 further comprising a microphone adapted to record sound waves.

5. The ECG module of claim 4 further comprising an audio processor adapted to receive the sound waves and convert the sound waves to digital audio signals of a user's voice

6. The ECG module of claim 1 further comprising a USB port for a wired connection to the electronic computer

7. The ECG module of claim 1 further comprising a Bluetooth radio adapted to communicate with a near field communication device of the electronic computer.

8. The ECG module of claim 7 wherein the electronic computer is a portable computing device.

9. The ECG module of claim 8 wherein the portable computing device delivers the ECG data to a server and a second electronic computer communicates with the server to receive the ECG data over the Internet.

10. A method of acquiring electrocardiograph (ECG) data, the method comprising the steps of:
connecting ECG electrodes to a patient;
receiving an extended duration of ECG data from the ECG electrodes at a module;
transmitting the ECG data to a network server of a network;
acquiring the ECG data at a device communicating with the network server;
reviewing the ECG data at the device; and
producing a report representative of the ECG data.

11. The method of claim 10 further comprising the steps of:
generating filtered ECG data from the raw ECG data at the module network server.

12. The method of claim 10 further comprising the steps of:
transmitting the ECG data from the module to an electronic computer via a communication protocol; and
viewing the ECG data at the electronic computer in real time.

13. The method of claim 12 wherein the communication protocol is near field communication.

14. The method of claim 12 wherein the electronic computer is a portable computing device.

15. The method of claim 10 further comprising the step of:
notifying a medical professional that ECG data has been transmitted to the network server.

16. The method of claim 10 further comprising the steps of:
receiving the ECG data from the network server; and
automatically identifying segments of the ECG data identifying a heart arrhythmia.

17. The method of claim 10 further comprising the step of:
modifying the report for the ECG data.

18. The method of claim 10 wherein the report includes a medical professional's interpretation of the ECG data.

19. The method of claim 10 wherein the network server stores previous ECG data of patient.

20. The method of claim 19 wherein the report includes comparison of previous and current ECG data.
